(19) United States
(12) Patent Application Publication
(10) Pub. No.: US 2021/0134417 A1
(43) Pub. Date: May 6, 2021

(54) INFORMATION PROCESSING APPARATUS, INFORMATION PROCESSING METHOD, AND INFORMATION PROCESSING PROGRAM

(71) Applicant: FUJIFILM CORPORATION, Tokyo (JP)

(72) Inventors: Takeshi FUKUDA, Kanagawa (JP); Haruyasu NAKATSUGAWA, Kanagawa (JP)

(21) Appl. No.: 17/079,558

(22) Filed: Oct. 26, 2020

(30) Foreign Application Priority Data
Nov. 6, 2019 (JP) 2019-201611

(51) Int. Cl. G16H 20/10; G16H 40/20; G16H 10/40; G16H 15/00

(52) U.S. Cl. CPC G16H 20/10; G16H 15/00; G16H 10/40; G16H 40/20

(57) ABSTRACT

An information processing apparatus predicts, on the basis of a current number of cases of a chronic disease in a veterinary hospital and the number of consumable items used during a predetermined period, which is the number of the consumable items used to examine an animal that is a subject suffering from the cases, a total number of the consumable items used during the period; and derives a shortage amount of the consumable items in the period on the basis of the predicted total number of the consumable items and a current stock number of the consumable items.

### CROSS-REFERENCE TO RELATED APPLICATIONS

The present application claims priority under 35 U.S.C. § 119 to Japanese Patent Application No. 2019-201611 filed on Nov. 6, 2019. The above application is hereby expressly incorporated by reference, in its entirety, into the present application.

### BACKGROUND

### 1. Technical Field

The present disclosure relates to an information processing apparatus, an information processing method, and n information processing program.

### 2. Description of the Related Art

There is disclosed a store-side apparatus that is connected to a plurality of hospital-side apparatuses, checks a stock status of medical products in a hospital, and determines whether it is necessary to place an additional order since the number of stocks is small (JP2002-041672A). The store-side apparatus transmits, to the hospital-side apparatus, notification information that it is desirable to place an additional order for a medical product for which it is determined that it is necessary to place the additional order, for example. Thus, the hospital that has received the notification information may place an order without delay for a medical product that is scheduled to be used.

### SUMMARY

However, in a hospital where a human is a subject, since an examination of a sample collected from the subject, such as a blood examination, is performed outside the hospital, it is not necessary for the hospital to manage stock of consumable items used in the examination. On the other hand, in a veterinary hospital where an animal is a subject, since an examination of a sample collected from the subject is performed in the hospital, it is necessary to manage stock of consumable items used in the examination at the veterinary hospital. In addition, it is necessary that the stock of the consumable items should be accurately managed.

However, in the technique disclosed in JP2002-041672A, stock of medical products in a hospital where a human is a subject is managed, and it is determined whether it is necessary to place an additional order depending on whether the number of medical products in stock is less than a predetermined number. Accordingly, in the technique disclosed in JP2002-041672A, there is a case where stock of consumable items used in examination may not be accurately managed.

The present disclosure has been made in view of the above circumstances, and provides an information processing apparatus, an information processing method, and an information processing program capable of accurately managing stock of consumable items used in examination of an animal.

In order to achieve the above object, according to an aspect of the present disclosure, there is provided an information processing apparatus comprising: a prediction unit that predicts, on the basis of a current number of cases of a chronic disease in a veterinary hospital and the number of consumable items used during a predetermined period, which is the number of the consumable items used to examine an animal that is a subject suffering from the cases, a total number of the consumable items used during the period; and a derivation unit that derives a shortage amount of the consumable items in the period on the basis of the total number of the consumable items predicted by the prediction unit and a current stock number of the consumable items.

The information processing apparatus according to the aspect of the present disclosure may further comprise a notification unit that notifies the shortage amount of the consumable items derived by the derivation unit.

Further, in the information processing apparatus according to the aspect of the present disclosure, the notification unit may further notify a warning in a case where the shortage amount of the consumable items is equal to or greater than a predetermined threshold value.

Further, in the information processing apparatus according to the aspect of the present disclosure, the notification unit may further notify information indicating that stocks are excessive in a case where the shortage amount is less than a predetermined second threshold value.

Further, the information processing apparatus according to the aspect of the present disclosure may further comprise an ordering unit that orders the consumable items in the shortage amount of the consumable items derived by the derivation unit.

Further, the information processing apparatus according to the aspect of the present disclosure may be a test kit used for examining a sample collected from the subject in the veterinary hospital.

Further, according to another aspect of the present disclosure, there is provided an information processing method executed by a computer, the method comprising: predicting, on the basis of a current number of cases of a chronic disease in a veterinary hospital and the number of consumable items used during a predetermined period, which is the number of the consumable items used to examine an animal that is a subject suffering from the cases, a total number of the consumable items used during the period; and deriving a shortage amount of the consumable items in the period on the basis of the predicted total number of the consumable items and a current stock number of the consumable items.

In addition, according to still another aspect of the present disclosure, there is provided an information processing program causing a computer to execute: a process of predicting, on the basis of a current number of cases of a chronic disease in a veterinary hospital and the number of consumable items used during a predetermined period, which is the number of the consumable items used to examine an animal that is a subject suffering from the cases, a total number of the consumable items used during the period; and a process of deriving a shortage amount of the consumable items in the period on the basis of the predicted total number of the consumable items and a current stock number of the consumable items.

Further, according to still another aspect of the present disclosure, there is provided an information processing apparatus comprising: a memory that stores a command executed by a computer; and a processor configured to execute the stored command, in which the processor predicts, on the basis of a current number of cases of a chronic disease in a veterinary hospital and the number of consumable items used during a predetermined period, which is the number of the consumable items used to examine an animal that is a subject suffering from the cases, a total number of the consumable items used during the period; and derives a shortage amount of the consumable items in the period on the basis of the predicted total number of the consumable items and a current stock number of the consumable items.

According to the present disclosure, it is possible to accurately manage stock of consumable items used in examination of an animal.

### BRIEF DESCRIPTION OF THE DRAWINGS

FIG. 1 is a block diagram showing an example of a hardware configuration of an information processing apparatus.

FIG. 2 is a diagram showing an example of stock data.

FIG. 3 is a block diagram showing an example of a functional configuration of the information processing apparatus.

FIG. 4 is a diagram for explaining a process of deriving a shortage amount of consumable items.

FIG. 5 is a diagram showing an example of a notification screen for notifying a shortage amount of consumable items.

FIG. 6 is a flowchart showing an example of a stock management process.

FIG. 7 is a block diagram showing an example of a functional configuration of an information processing apparatus according to a modification example.

FIG. 8 is a diagram for explaining an example of comparing a total number of used consumable items with that of another hospital.

### DETAILED DESCRIPTION

Hereinafter, an example for carrying out the technique of the present disclosure will be described in detail with reference to the accompanying drawings. It should be noted that in the following embodiments, an example in which a dog is applied as an animal that is a subject will be described. In addition, the term “animal” in the present specification means animals such as dogs and cats, excluding “humans”.

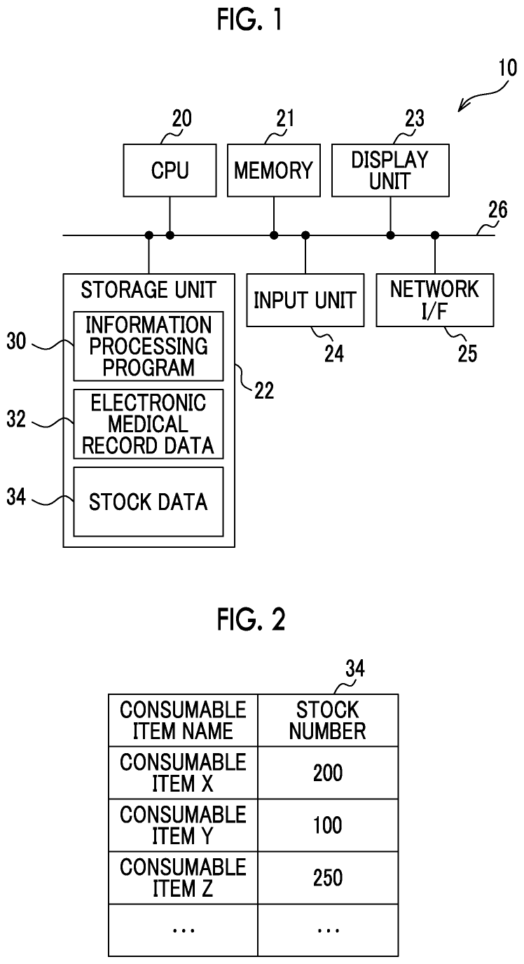

First, a hardware configuration of an information processing apparatus 10 according to the present embodiment will be described with reference to FIG. 1. The information processing apparatus 10 is provided in, for example, a veterinary hospital. Examples of the information processing apparatus 10 include a personal computer, a server computer, or the like. The information processing apparatus 10 may be a cloud server.

As shown in FIG. 1, the information processing apparatus 10 includes a central processing unit (CPU) 20, a memory 21 as a temporary storage area, and a non-volatile storage unit 22. Further, the information processing apparatus 10 includes a display unit 23 such as a liquid crystal display, an input unit 24 such as a keyboard and a mouse, and a network interface (I/F) 25 connected to a network. The CPU 20, the memory 21, the storage unit 22, the display unit 23, the input unit 24, and the network I/F 25 are connected to a bus 26.

The storage unit 22 is realized by a hard disk drive (HDD), a solid state drive (SSD), a flash memory, or the like. An information processing program 30 is stored in the storage unit 22 that is a storage medium. The CPU 20 reads out the information processing program 30 from the storage unit 22, develops the information processing program 30 in the memory 21, and executes the developed information processing program 30.

Further, the storage unit 22 stores electronic medical record data 32 including data used for an electronic medical record of a veterinary hospital. The electronic medical record data 32 includes, for each subject, identification information of the subject, a name of a disease with which the subject is affected, a visit date of the subject, and the number of consumable items used for examination.

Further, the storage unit 22 stores stock data 34 for stock management of consumable items. FIG. 2 shows an example of the stock data 34. As shown in FIG. 2, in the stock data 34, each consumable item and a current stock number of the consumable item are associated with each other. In the present embodiment, an example in which a test kit used for examining a sample collected from a subject in a veterinary hospital is applied as a consumable item will be described. Further, in the present embodiment, the consumable item is used for an examination for diagnosing a chronic disease in the subject. In the present embodiment, an example in which a chronic kidney disease is applied as the chronic disease will be described, but the present invention is not limited thereto, and chronic diseases other than the chronic kidney disease may be applied. Examples of the sample collected from the subject include urine, blood, and the like, and examples of the test kit include a test kit for measuring creatinine in urine, and the like. The current stock number in the stock data 34 is updated by a user in accordance with usage and replenishment of consumable items.

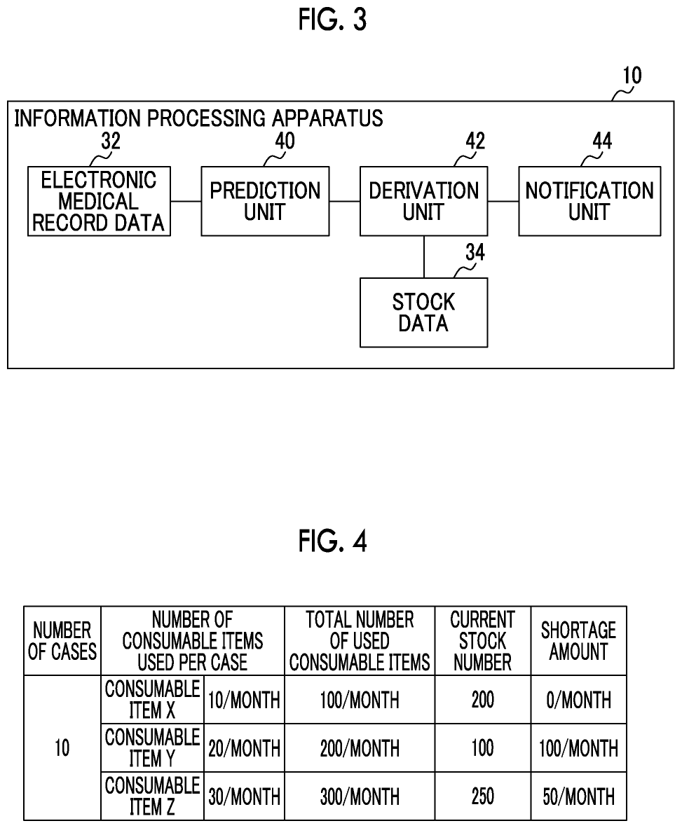

Next, a functional configuration of the information processing apparatus 10 according to the present embodiment will be described with reference to FIG. 3. As shown in FIG. 3, the information processing apparatus 10 includes a prediction unit 40, a derivation unit 42, and a notification unit 44. The CPU 20 executes the information processing program 30 to function as the prediction unit 40, the derivation unit 42, and the notification unit 44.

The prediction unit 40 predicts a total number of consumable items used in a predetermined period T1, on the basis of the number of current cases of a chronic disease in a veterinary hospital, and the number of consumable items used in the period T1, which is the number of consumable items used to examine an animal that is a subject suffering from the above cases. Details of a prediction process performed by the prediction unit 40 will be described with reference to FIG. 4. In the present embodiment, a case where one month is applied as the period T1 will be described, but the present invention is not limited thereto, and one week may be applied or two months may be applied. The period T1 is determined in advance, for example, according to the frequency of ordering consumable items. In this embodiment, an example will be described in which consumable items are ordered once a month that is the same period as the period T1.

First, the prediction unit 40 counts the number of subjects who have suffered from the chronic kidney disease and came to the veterinary hospital during the latest period T1 with reference to the electronic medical record data 32, to thereby derive the number of current cases of the chronic kidney disease.

Then, the prediction unit 40 analyzes the electronic medical record data 32 to derive the number of consumable items used in the period T1, that is, the number of consumable items used to examine the subject suffering from the chronic kidney disease. Specifically, the prediction unit 40 derives an average value of the number of consumable items used in the latest period T1 for each derived case, to thereby derive the number of consumable items per case used in the period T1. In the example of FIG. 4, with respect to one case, 10 consumable items X are used per month, 20 consumable items Y are used per month, and 30 consumable items Z are used per month.

Then, the prediction unit 40 multiplies the number of current cases of the chronic kidney disease by the number of consumable items used in the derived period T1, to thereby derive a total number of consumable items used in the period T1 for each consumable item. The derived total number of consumable items is a prediction value of the total number of consumable items predicted by the prediction unit 40. In the example of FIG. 4, the total number of the consumable items X used for the examination of the chronic kidney disease for one month is 100 (=10×10), the total number of the consumable items Y is 200 (=10×20), and the total number of the consumable items Z is 300 (=10×30).

The derivation unit 42 derives a shortage amount of the consumable items in the period T1 on the basis of a total number of consumable items predicted by the prediction unit 40 and a current stock number of the consumable items. Specifically, as shown in FIG. 4, the derivation unit 42 subtracts the current stock number of the consumable items from the total number of the consumable items predicted by the prediction unit 40 with reference to the stock data 34, to thereby derive the shortage amount of the consumable items in the period T1. In the example of FIG. 4, the shortage amount of the consumable items X in one month is 0 (=100−200), the shortage amount of the consumable items Y is 100 (=200−100), and the shortage amount of the consumable items Z is 50 (=300−250). As shown in FIG. 4, in the present embodiment, in a case where the shortage amount derived by the derivation unit 42 is 0 or less, it is considered that the consumable items are not insufficient, and thus, the shortage amount is set to 0. The derivation unit 42 may derive the shortage amount of the consumable items in consideration of a predetermined margin.

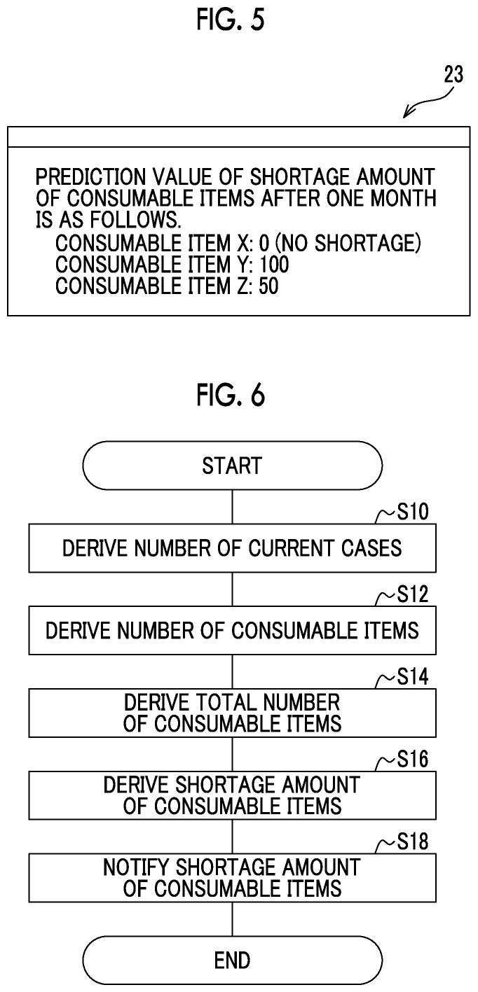

The notification unit 44 notifies the shortage amount of the consumable items derived by the derivation unit 42. In the present embodiment, the notification unit 44 performs the notification by displaying the shortage amount of the consumable items derived by the derivation unit 42 on the display unit 23. As an example, a notification screen shown in FIG. 5 is displayed on the display unit 23, by the notification by the notification unit 44. The notification unit 44 may notify the shortage amount of the consumable items derived by the derivation unit 42 by e-mail or the like, instead of the display on the display unit 23.

Further, for example, the notification unit 44 may further notify a warning in a case where the shortage amount of the consumable items is equal to or greater than a predetermined first threshold value. In this case, a configuration in which the notification unit 44 further displays, on the display unit 23, a warning message for prompting a user to order a consumable item that is predicted to be insufficient in a case where the shortage amount of the consumable items derived by the derivation unit 42 is one or more is exemplified.

Further, for example, the notification unit 44 may further notify information indicating that the stock is excessive in a case where the shortage amount of the consumable items is smaller than a predetermined second threshold value. In this case, a configuration in which, in a case where a value obtained by subtracting the current stock number of the consumable items from the total number of the consumable items predicted by the prediction unit 40 is smaller than −100, the notification unit 44 further displays a message for notifying that the stock is excessive on the display unit 23 is exemplified.

Next, an operation of the information processing apparatus 10 according to the present embodiment will be described with reference to FIG. 6. As the CPU 20 executes the information processing program 30, a stock management process shown in FIG. 6 is executed. The stock management process is executed at regular timings such as once every period T1, for example.

In Step S10 of FIG. 6, the prediction unit 40 counts the number of subjects that have suffered from the chronic kidney disease and have come to the veterinary hospital in the most recent month with reference to the electronic medical record data 32, to thereby derive the number of current cases of the chronic kidney disease. In Step S12, the prediction unit 40 analyzes the electronic medical record data 32 and derives the number of consumable items used in the period T1, which is the number of the consumable items used to examine the subject suffering from the chronic kidney disease, as described above.

In Step S14, the prediction unit 40 multiplies the number of the current cases of the chronic kidney disease derived in Step S10 by the number of the consumable items used in the period T1 derived in Step S12 to thereby derive a total number of the consumable items used in the period T1 for each consumable item, as described above. In Step S16, the derivation unit 42 derives a shortage amount of the consumable items in the period T1 on the basis of the total number of the consumable items derived in Step S14 and a current stock number of the consumable items, as described above.

In Step S18, the notification unit 44 notifies the shortage amount of the consumable items derived in Step S16, as described above. In a case where the process of Step S18 ends, the stock management process ends.

As described above, according to the present embodiment, the total number of the consumable items used in the period T1 is predicted on the basis of the current number of cases of a chronic disease in a veterinary hospital and the number of the consumable items used in the period T1, that is, the number of the consumable items used to examine an animal that is a subject suffering from the cases. Further, according to the present embodiment, the shortage amount of the consumable items in the period T1 is derived on the basis of the predicted total number of the consumable items and the current stock number of the consumable items.

In the case of a chronic disease, there is a high possibility that a subject visits a hospital on a regular basis, and also, there is a high possibility that the number of consumable items used to examine the chronic disease is used on a regular basis. Therefore, according to the present embodiment, it is possible to accurately derive a shortage amount of the consumable items used to examine the chronic disease, and as a result, it is possible to accurately manage the stock of the consumable items used to examine an animal.

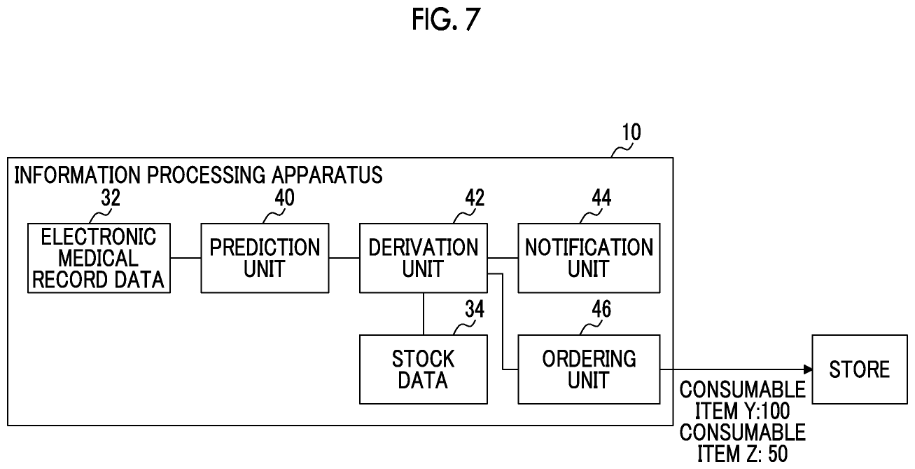

In the above-described embodiment, as shown in FIG. 7 as an example, the information processing apparatus 10 may further comprise an ordering unit 46 that orders a consumable item in the shortage amount of the consumable items derived by the derivation unit 42. In this case, the ordering unit 46 transmits, for example, an order instruction for ordering the consumable items for the shortage amount of the consumable items derived by the derivation unit 42 to a store that sells the consumable items. In this embodiment, the CPU 20 executes the information processing program 30 to further function as the ordering unit 46. Further, in this embodiment, the information processing apparatus 10 may not comprise the notification unit 44.

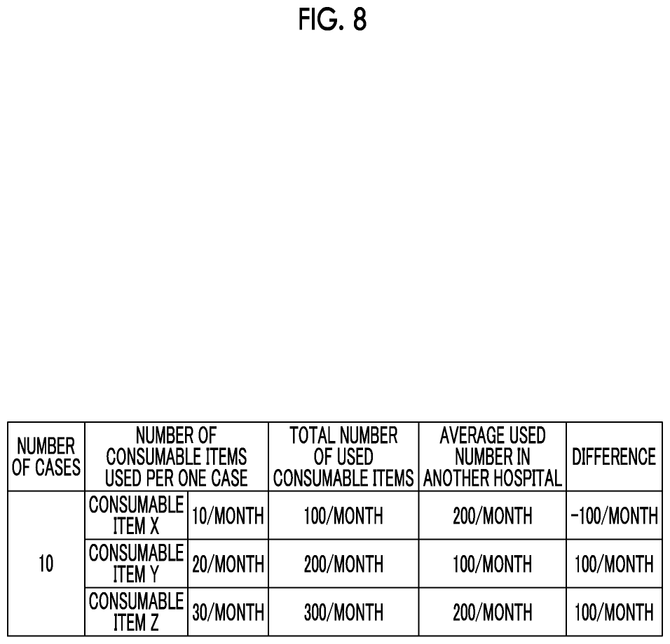

Further, in the above-described embodiment, as shown in FIG. 8 as an example, the total number of the consumable items used in the period T1 predicted by the prediction unit 40 may be compared with an average value of the total number of the consumable items used in another veterinary hospital in the period T1. FIG. 8 shows an example in which the total number of the consumable items X used in the period T1 is smaller by 100, and the total number of the consumable items Y and the total number of the consumable items Z are respectively larger by 100, compared with another veterinary hospital. From this comparison result, a manager of a veterinary hospital may take measures, for example, to carry out a campaign to use more consumable items that are used less than another veterinary hospital.

Further, in the above-described embodiment, a case where a dog is applied as an animal that is a subject has been described, but the present invention is not limited thereto. As the animal that is the subject, for example, an animal other than the dog, such as a cat, may be applied.

Further, in the above embodiment, for example, as a hardware structure of a processing unit (processing unit) that executes various processes, such as the prediction unit 40, the derivation unit 42, and the notification unit 44, the following various processors may be used. As described above, the various processors include, in addition to the CPU that is a general-purpose processor that executes software (programs) to function as various processing units, a programmable logic device (PLD) that is a processor of which a circuit configuration is changeable after manufacturing, such as a field programmable gate array (FPGA), a dedicated electrical circuit that is a processor having a circuit configuration specifically designed to execute a specific process, such as an application specific integrated circuit (ASIC).

One processing unit may be configured by one of these various processors, or may be configured by a combination of two or more processors of the same type or different types (for example, a combination of a plurality of FPGAs or a combination of a CPU and an FPGA). Further, a plurality of processing units may be configured by one processor.

As an example in which the plurality of processing units is configured by one processor, first, as represented by a computer such as a client and a server, there is a configuration in which one processor is configured by a combination of one or more CPUs and software and the processor functions as a plurality of processing units. Second, as represented by a system on chip (SoC) or the like, there is a configuration in which a processor that realizes the functions of the entire system including a plurality of processing units by one integrated circuit (IC) chip is used. As described above, the various processing units are configured using one or more of the above various processors as a hardware structure.

Further, as a hardware structure of these various processors, more specifically, electric circuitry in which circuit elements such as semiconductor elements are combined may be used.

Further, in the above-described embodiment, a configuration in which the information processing program 30 is stored (installed) in the storage unit 22 in advance has been described, but the invention is not limited thereto. The information processing program 30 may be provided in a form of being recorded on a recording medium such as a compact disc read only memory (CD-ROM), a digital versatile disc read only memory (DVD-ROM), a universal serial bus (USB) memory, or the like. Further, the information processing program 30 may be downloaded from an external device through a network.

What is claimed is:

1. An information processing apparatus comprising:
a prediction unit that predicts, on the basis of a current number of cases of a chronic disease in a veterinary hospital and the number of consumable items used during a predetermined period, which is the number of the consumable items used to examine an animal that is a subject suffering from the cases, a total number of the consumable items used during the period; and
a derivation unit that derives a shortage amount of the consumable items in the period on the basis of the total number of the consumable items predicted by the prediction unit and a current stock number of the consumable items.

2. The information processing apparatus according to claim 1, further comprising:
a notification unit that notifies the shortage amount of the consumable items derived by the derivation unit.

3. The information processing apparatus according to claim 2,
wherein the notification unit further notifies a warning in a case where the shortage amount of the consumable items is equal to or greater than a predetermined threshold value.

4. The information processing apparatus according to claim 2,
wherein the notification unit further notifies information indicating that stocks are excessive in a case where the shortage amount is less than a predetermined second threshold value.

5. The information processing apparatus according to claim 1, further comprising:
an ordering unit that orders the consumable items in the shortage amount of the consumable items derived by the derivation unit.

6. The information processing apparatus according to claim 1,
wherein the consumable items are a test kit used for examining a sample collected from the subject in the veterinary hospital.

7. An information processing method executed by a computer, the method comprising:
predicting, on the basis of a current number of cases of a chronic disease in a veterinary hospital and the number of consumable items used during a predetermined period, which is the number of the consumable items used to examine an animal that is a subject suffering from the cases, a total number of the consumable items used during the period; and
deriving a shortage amount of the consumable items in the period on the basis of the predicted total number of the consumable items and a current stock number of the consumable items.

8. A non-transitory computer-readable storage medium storing an information processing program causing a computer to execute:
a process of predicting, on the basis of a current number of cases of a chronic disease in a veterinary hospital and the number of consumable items used during a predetermined period, which is the number of the consumable items used to examine an animal that is a subject suffering from the cases, a total number of the consumable items used during the period; and
a process of deriving a shortage amount of the consumable items in the period on the basis of the predicted total number of the consumable items and a current stock number of the consumable items.
